(19) United States
(12) Patent Application Publication
(10) Pub. No.: US 2010/0166806 A1
(43) Pub. Date: Jul. 1, 2010

(54) Combination therapy comprising the use of protein kinase C modulators and Histone Deacetylase inhibitors for treating HIV-1 latency

(75) Inventor: Trevor Percival Castor, Arlington, MA (US)

Correspondence Address: Dr. Trevor P. Castor, 3-E Gill Street, Woburn, MA, 01801, US

(73) Assignee: Aphios Corporation, Woburn, MA (US)

(21) Appl. No.: 12/317,758

(22) Filed: Dec. 29, 2008

(51) Int. Cl. A61K 9/00; A61K 31/366; A61K 31/19; A61P 31/18; C07D 493/22; A61K 31/165

(52) U.S. Cl. 424/400; 514/450; 514/557; 549/267; 514/575

(57) ABSTRACT

The invention relates to a combination of treatments, more particularly a combination treatment for HIV-1 infection. The present invention is directed to the use of bryostatin-1 and their natural and synthetic derivatives for AIDS therapy, in particular to the use of bryostatins in combination with other active drugs such as Histone Deacetylases (HDACs) inhibitors and anti-retrovirals, for the treatment of HIV-1 latency. According to the present invention, we provide a combination therapy for the treatment of HIV-1 latency which employs bryostatin-1 (and analogues) and one of the following HDAC inhibitors; valproic acid, butyrate derivatives, hydroxamic acids and benzamides. While HDACi can be used in continuous dosing protocol, bryostatins can be used following a cyclical dosing protocol. Bryostatins can be formulated in pharmaceutical acceptable carriers including nanoparticles, phospholipids nanosomes and/or biodegradable polymer nanospheres. This combination therapy needs to be used in patients treated with antiretroviral therapy (HIV-1 protease inhibitors, HIV-1 reverse transcriptase inhibitors, HIV-1 integrase inhibitors, CCR5 co-receptor inhibitors and fusion inhibitors).

This invention relates to a combination of treatments, more particularly a combination treatment for HIV-1 infection and latency.

### FIELD OF THE INVENTION

The present invention is directed to the use of bryostatin-1 and their natural and synthetic derivatives for AIDS therapy, in particular to the use of protein kinase C modulators such as bryostatins in combination with other active drugs such as Histone Deacetylases (HDACs) inhibitors and antiretrovirals, for the treatment of HIV-1 latency.

### BACKGROUND OF THE INVENTION

HIV infects several cell types during the course of infection and progression to acquired immune deficiency syndrome (AIDS). The persistence of latent HIV-infected cellular reservoirs represents the major hurdle to virus eradication with highly active anti-retroviral therapy (HAART), since latently infected cells remain a permanent source of viral reactivation. As a result, a sudden rebound of the virus load after interruption of HAART is generally observed. The HIV-virus establishes a persistent infection in CD4+ T lymphocytes (and to a lesser extent in macrophages as well), creating a persistent reservoir consisting mainly of latently infected resting memory CD4+ T cells. Although pre- and post-integration latencies have been described in HIV-1, the reservoir that appears to be the major barrier to eradication is composed of latently infected cells carrying an integrated provirus that is transcriptionally silent. The extremely long half-life of these cells, combined with a tight control of HIV-1 expression, has been reported to make this reservoir ideally suited to maintain hidden copies of the virus, eventually triggering a novel systemic infection upon discontinuation of therapy.

The current therapies directed against viral proteins (HAART) have been problematic because of long-term toxicity, inhibitor resistance, and the inability to target persistent reservoirs. Therefore, it has been suggested that reactivation of the latent reservoirs could allow effective targeting and possible eradication of the virus. Immunoactivation therapy to reduce the latent pool of HIV by treatment with the anti-CD3 antibody OKT-3 alone or in combination with interleukin-2, substantially failed to significantly decrease the viral reservoir.

Nevertheless, a host of small molecules including phorbol esters, ingenols and 1,2-diacylglycerol analogs, has been suggested as agents to reactivate HIV and eradicate the pool of latently HIV-infected CD4⁺ T cells. More recently, non-tumor-promoting phorbol deoxyphorbol esters such as prostratin have been directly evaluated for their ability to reactivate latent virus both in latently infected cell lines and in primary memory T cells from HIV infected patients. Prostratin and other non-tumorogenic PKC-activators reactivates HIV-1 latency in “vitro” by signalling through both the ERK and the PKC pathways. Moreover, the PKC agonists (prostratin and Ingenol-3-angelate) also down-regulates the expression of the HIV-1 receptor CD4and the co-receptor CXCR4, thus avoiding the new infection of CD4^{+ cells.}

The capacity of prostratin to behave as an in vivo agent to purge latent HIV-1 proviruses has raised considerable interest, owing to a potential clinical application in combination with HAART to eradicate HIV-1 infection. However, relatively high concentrations of prostratin are required to reactivate HIV-1 latency and it has been suggested that prostratin may have negative side effects and therefore it is unlikely that high-doses or long term treatment would be well tolerated. Since the PKC-dependent activation of the NF-κB and ERK pathways are well known mechanisms to reactivate HIV-1 latency, the identification of novel PKC activators lacking tumor-promoter activity such as bryostatins are of special interest for the clinical development of drugs that antagonize HIV-1 latency. In fact, bryostatin-1 is currently undergoing several clinical trials against cancer malignances.

The bryostatins are a structurally novel family of marine macrolides isolated from the bryozoan invertebrates Bugula neritina Linnaeus and Amathia convulata. Eighteen bryostatins have so far been isolated from these two organisms. All bryostatins possess a 20-membered macrolactone in which there are three remotely-functionalised pyran rings interconnected by an (E)-disubstituted alkene and a methylene bridge; all family members also contain a pair of geminal dimethyls at C(8) and C(18); each bryostatin has a four-carbon side-chain emanating from its A and C-rings, and virtually all have an exocyclic methyl enoate in their B and C rings. Bryostatin 1 shows remarkable in vitro and in vivo anticancer effects against a range of tumours. Bryostatin 1 has recently completed several anticancer trials in man where its most significant side effect was mylagia. The trials clearly demonstrated that bryostatin 1 has considerable potential for the treatment of ovarian and relapsed low-grade non-Hodgkin's lymphoma, it being effective when given alone, or in combination with other anticancer drugs. The antitumour effects of bryostatin 1 have been linked to its ability to selectively modulate the functioning of various individual protein kinase C (PKC) isozymes within cells. Bryostatin 1 competitively binds to the phorbol ester-diacylglycerol binding sites of PKC isozymes. The PKC family of serine/threonine kinases plays a central role in mediating the signal transduction of extracellular stimuli, which result in the production of the second messenger 1,2-diacyl-sn-glycerol (DAG). PKC is also the primary target of the phorbol esters, ingenols, DAG-lactones and bryostatins and consists of a family of 12 members that are classified into three major subfamilies. The classical PKCs (α, β_{I}, β_{II} and γ) are Ca²⁺- and DAG-dependent, whereas the novel PKCs (δ, ε, η and θ) are Ca²⁺-independent but DAG-responsive. The atypical PKCs (ζ and λ/l) lack the responses to both Ca²⁺ and DAG (Newton, 2001). A highly conserved cysteine-rich motif (the so-called “C1 domain”) in the regulatory region of the PKCs acts as the specific receptor for the DAG signal. The cPKCs and nPKCs have two tandem C1 domains in their N-terminal domain, the C1a and C1b domains, which show high binding affinities in vitro for DAG, phorbol esters and other PKC activators such as Ingenol and bryostatin-1.

The translocation of PKCs from cytoplasm to plasma membrane and other subcellular localizations is the hallmark for PKC activation, and isozyme-specific functions may result in part from a different subcellular localization of the activated enzyme. Several studies have shown that the translocation of PKC is isoform-, cell type-, and activator-specific, and, for phorboids endowed with tumor promoter activity is tightly regulated by lipophilicity. There is a general agreement that only PKC agonists inducing a sustained PKC translocation to the cell membrane are endowed with tumor promoter activity. It has been proposed that the protective action of bryostatin 1 upon some PKCs might be the result of it inducing a “stabilising” conformational change in these enzymes, preventing them from inserting into the plasma membrane. Clearly, the identification of potent natural or synthetic PKC agonists lacking tumor-promoter activities has opened new research avenues for the treatment of HIV-1 latency. Moreover, bryostatin-1 has been shown to down-regulate the expression of CD4 antigen, which is the main receptor for HIV-1 entry into the cells. Reactivation of HIV-1 latency in T cells required cell activation and it have been demonstrated that bryostatin-1 activates resting humans T cells. However the effect of bryostatins on HIV-1 reactivation in human T cells was never investigated.

Histone deacetylases and histone acetyltransferases (HATs) are two opposing groups of enzymes involved in chromatin remodeling by modifying the acetylation states of histones. HATs catalyze histone acetylation on the amino groups of lysine residues in the N-terminal tails of core histones. Neutralization of positive charge and increase in hydrophobicity by histone acetylation greatly reduce the affinity of histone for DNA template, thus altering nucleosome structure, facilitating the binding of transcription factors to nucleosomal DNA, and enhancing transcription. On the contrary, histone deacetylases (HDACs) catalyze deacetylation by cleaving acetyl groups, resulting in tightening of nucleosomal integrity, restriction of the access of transcription factors, and suppression of transcription. Since the discovery of the first HDAC in 1996, at least 18 members have been identified. Mammalian HDACs can be categorized into three classes based on sequence homology to yeast counterparts. Class I includes HDAC 1, 2, 3, and 8 mostly localized to the nucleus with ubiquitous distribution throughout human cell lines and tissues. Class II HDACs, which can be further categorized into two subclasses, IIa (HDAC 4, 5, 7, and 9) and IIb (HDAC 6 and 10) and can shuttle between the cytoplasm and nucleus with likely tissue-specific distribution.

To date, several structurally distinct classes of HDAC inhibitors have advanced into Phase I and/or II clinical trials in solid tumors and hematological malignancies. On the basis of their chemical structures, major HDAC inhibitors can be classified into four categories: short-chain fatty acids (butyrate, valproate and phenylbutirate), hydroxamic acids (Trichostatin A and suberoylanilide hydroxamic acid and LAQ-824), benzamide derivatives (MS-275 and CI-944), and cyclic peptides.

The regulation of transcription of the human immunodeficiency virus (HIV) is a complex event that requires the cooperative action of both viral and cellular components. In latently infected resting CD4+ T cells, HIV-1 transcription seems to be repressed by deacetylation events mediated by histone deacetylases (HDACs). The HIV-1 provirus is packaged into chromatin whereby, independently of the site of integration, nucleosomes are positioned precisely on the 50-LTR promoter region with respect to cis-acting regulatory elements. This higher ordered chromatin structure negatively regulates gene expression by restricting access of the transcriptional machinery to the viral promoter. Two nucleosomes (called nuc-0 and nuc-1) are positioned within the viral promoter. Importantly, upon stimulation with histone deacetylase inhibitors, nuc-1 becomes rapidly and specifically disrupted by acetylation of specific lysine residues within histone H3 and H4 of present in this nucleosome. This may be mediated through a mechanism which involves a displacement of corepressor complexes containing HDAC, which has been recruited to the viral promoter by host factors such as LSF with YY1 and the NF-kB p50 homodimers, in response to the recruitment of chromatin remodeling and modifying complexes by NF-kB p50/p65 heterodimers (or Tat). Indeed, it have been demonstrated deacetylase inhibitors (HDACi) (such as trichostatin A (TSA), trapoxin (TPX), valproic acid (VPA) and sodium butyrate (NaBut) induce the transcriptional activation of the HIV-1 promoter. This occurs in ex vivo transiently or stably transfected HIV-1 LTR promoter reporter constructs, in latently HIV-1-infected cell lines, on in vitro chromatin-reconstituted HIV-1 templates, as well as in the context of a de novo infection. Therefore, it is generally accepted that the use of deacetylases inhibitors in the treatment of HIV infection may represent a valuable approach for purging the latently infected reservoirs in HAART-treated individuals. A recent proof-of-concept study has shown that valproic acid induced a significant depletion of HIV-1 latent infected cells in three of four patients included in the study. In this study the VPA dose of 500-750 mg twice at day was adjusted to maintain plasma concentrations within a defined range (50-100 mg/L). However, other clinical studies failed to demonstrate a decline in the HIV-1 reservoir and conclude that the clinical use of VA has no ancillary effect on the decay of the latent reservoir. Besides valproic acid there are no clinical reports regarding the use of other HDACs inhibitor for the treatment of HIV-1 latency. In summary it is likely that single therapy with HDACs inhibitors will not be sufficient to reactivate latent HIV-1 from the patient viral reservoir and a more potent therapy will be required to purge HIV-1 in patients.

### SUMMARY OF THE INVENTION

According to the present invention, we provide a combination therapy for the treatment of HIV-1 latency which employs bryostatin-1 (and analogues) and one of the following HDAC inhibitors; valproic acid, butyrate derivatives, hydroxamic acids and benzamides. While HDACi can be used in continuous dosing protocol, bryostatins can be used following a cyclical dosing protocol. This combination therapy needs to be used in patients treated with antiretroviral therapy (HIV-1 protease inhibitors, HIV-1 reverse transcriptase inhibitors, HIV-1 integrase inhibitors, CCR5 co-receptor inhibitors and fusion inhibitors).

From our experiments using a specific and suitable model for HIV-1 reactivation we determined that a combination of bryostatins and HDACs inhibitors synergise to antagonise HIV-1 latency. This finding is essential for the formulation of the combination therapy using low concentrations of bryostatins. Accordingly, it is an object of the present invention to provide a potent anti-HIV-1 latency combination therapy with minimal adverse toxicological properties. Typical dosing protocols for the combination therapy are provided but not restricted. We further provide evidence that bryostatin-1 downregulates the expression of the HIV-1 receptors CD4 and CXCR4 and prevent HIV-1-induced cytotoxicity, which is mediated by the viral entry into the target cells. The effect of bryostatin-1 on HIV-1 receptors downregulation is not affected by the presence of valproic acid.

Various other objects and advantages of the present invention will become apparent to one skilled in the art from the drawings and the following description of the invention.

### BRIEF DESCRIPTION OF THE DRAWINGS

FIG. 1 shows chemical structures of different bryostatins.

FIG. 2 shows the HPLC profile of isolated bryostatin-1.

FIG. 3 shows the HPLC profile of isolated bryostatin-2.

FIG. 4 shows the HPLC profile of isolated bryostatin-3.

FIG. 5 shows the effect of Bryostatin-1 (100 nM) on HIV-1 reactivation.

FIG. 6 shows the effects of different bryostatins on HIV-1 reactivation.

FIG. 7 shows that bryostatin-1 is more potent than prostratin to reactivate HIV-1 latency

FIG. 8 shows the differential effects of increasing concentrations of bryostatin-1 and prostratin on the NF-kB pathway and the MAPKs (ERK and JNK) pathway.

FIG. 9 shows that classical PKCs are involved in bryostatin-1-induced HIV-1 reactivation.

FIG. 10 shows that HDACs inhibitors (Valproic acid [VPA] and Trychostatin [TSA]) synergise with a suboptimal concentration of bryostatin-1 (1 nM) to reactive HIV-1 latency.

FIG. 11 shows the synergistic effects of VPA (1 and 5 mM) and TSA (100 and 200 nM) with different concentrations of bryostatin-1 (1 and 10 nM) to reactive HIV-1 latency.

FIG. 12 shows the effect of bryostatin-1 on HIV-1 receptor expression in T cells.

FIG. 13 shows the cytoprotective effects of bryostatins on HIV-1-induced cell death.

### DETAILED DESCRIPTION OF THE INVENTION

Developing drugs directed against different targets of the HIV cycle is urgently needed, especially the development of drugs able to diminish or eradicate latent reservoirs. To this end, chemical modifications of known active compounds and the harnessing of natural resources is crucial for the improvement of drug strength and the reduction and elimination of potential toxicities. Clearly, the identification of potent natural or synthetic PKC agonists lacking tumor-promoter activities has opened new research avenues for the treatment of HIV-1 latency.

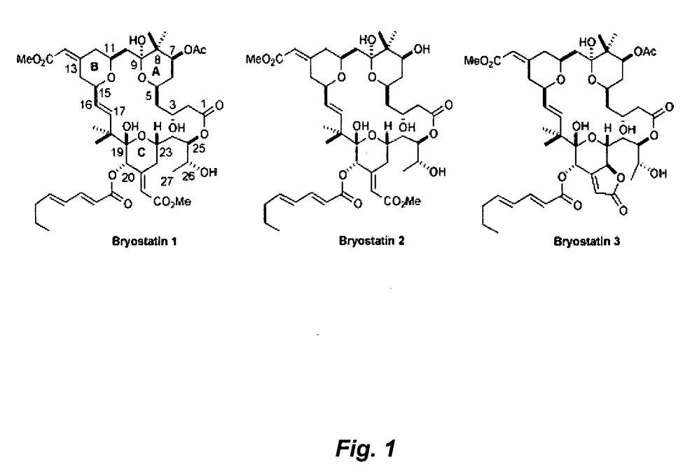

The present invention relates to an antiviral composition. The composition of the present invention comprises as the active ingredients, the marine macrocyclic lactones bryostatins in combination with one HDACs inhibitor and a pharmaceutically acceptable carrier. The bryostatin-1 of the present composition may be purified from a natural source or may be synthetically made. Bryostatin-1, -2, and -3 are natural compounds represented by the formulas shown in FIG. 1.

The very low number of latently HIV-infected cells in vivo makes purification and biochemical analysis of these cells impractical. As an experimentally tractable and relevant model of postintegration HIV latency, we have employed the Jurkat-LAT-GFP clone to explore HIV latency antagonising effects of bryostatins alone or in combination with distinct HDACs inhibitors. Jurkat-LAT-GFP cells contain a single, full-length integrated HIV provirus in which GFP has been substituted for Nef. This substitution allows rapid assessment of HIV transcriptional activity by cytometric detection of GFP epifluorescence. Similar models of HIV-1 latency have been used to study the effects of trychostatin A and TNFα on HIV-1 reactivation. Using this in vitro model to study HIV-1 reactivation it can be predicted the activity of bryostatins and HDACs inhibitors in humans. We show for the first time that bryostatins (1, 2 and 3) strongly induce HIV-1 reactivation at the concentration of 10 nM, which is in the range of plasma concentrations detected in humans treated with cycling dosing protocols of bryostatin-1.

The primary interest in Bryostatins has been initiated by recognition of the potent antiproliferative effects against various tumour cells. Such effects have been related to the ability of Bryo-1 to modulate protein kinase C (PKC) activity by activating or degrading certain isoforms of PKC. Since PKC activation mediates different signal pathways that in turn activate the transcription of latent HIV-1, we preincubated Jurkat-LAT-GFP cells with medium or the chemical inhibitors Gö6976 (classical PKCs inhibitor), Gö66850 (classical and novel PKCs inhibitor), Gö6983 (pan-PKC inhibitor), rottlerin (PKCδ inhibitor) and PD98059 (MEK inhibitor) at effective concentrations. Gö6976, Gö6850 and Gö6983 strongly inhibited GFP expression induced by Bryostatin-1 further implicating a PKC-dependent signaling step in this response. PD98059 partially inhibited SJ23B-induced HIV-1 reactivation suggesting that the ERK pathway is also activated by bryostatin-1. In contrast, the PKCδ inhibitor rottlerin did not affect phorbol-induced GFP expression, ruling out the involvement of this PKC in HIV-1 reactivation in Jurkat-LAT-GFP cells.

Since the experiments with the relatively specific PKCs inhibitors suggested that bryostatin-1 re-activates HIV-1 latency thorough the PKC pathway, we investigated biochemical targets downstream of PKC. Jurkat-LAT-GFP cells were stimulated with increasing concentrations of bryostatin-1 and the phosphorylation and degradation of the NF-κB inhibitor Iκbα, and the phosphorylation (activation) of the MAPKs, ERK and JNK, were investigated by western blots using specific mAbs. Bryostatin-1 induced phosphorylation and degradation of IεBα, and also the activation of the MAPKs, ERK1+2 and JNK1+2 in a concentration dependent manner. Importantly, our results show that bryostatin-1 at the concentration of 10 nM does not induce IκBα phosphorylation and degradation and JNK activation, but fully reactivates HIV-1 latency. Therefore, the therapeutic activity of bryostatin-1 for HIV-1 latency can be achieved at concentrations that do not activate signal transduction pathways (i.e. NF-κB and AP-1) that may result in negative side effects.

In addition to its HIV-1-latency antagonizing activity, bryostatin-1 also downregulates, at 10 nM concentration, the expression of the human HIV-1 receptors CD4and CXCR4 and prevents de novo HIV-1 infection as measured by virus-induced cytoxicity assays (EC₅₀ of 26 nM).

In another set of experiments we demonstrate that bryostatin-1 synergises with HDACs inhibitors (Valproic acid and TSA) to antagonise HIV-1 latency. HDACs inhibitors alone do not significantly reactivate HIV-1 latency but allow reducing the concentration of bryostatin-1 (at least one order of magnitude). Bryostatin-1 at 1 nM concentration can induce HIV-1 reactivation in the presence of therapeutically relevant concentrations of valproic acid. Thus, the therapeutic activity of bryostatin-1 can be drastically reduced in humans including a HDACs inhibitor in the combination therapy.

Another potential benefit of the combination therapy using bryostatin-1 and HDACs inhibitors for the treatment of HIV-1 latency can be inferred from the other published documents. Tumour necrosis factor-α have been shown to be release after bryostatin 1 injection in humans and tumour necrosis factor-α synergise with HDACs inhibitors to reactivate HIV-1 latency. Pharmacokinetic experiments have shown that after i.v. injection bryostatin-1 is accumulated in several tissues including lymph nodes and the gastrointestinal tract that represent potential organs harbouring HIV-1 latent infected cells. This represents another advantage for the use of bryostatins in the treatment of viral reservoirs.

It is expected that a combination therapy including bryostatins and HDACs inhibitors can purge the latent HIV-1 from the body but at least three mechanisms; 1) the reactivated virus will induce the death of the harboring cells and the emerging virus can not infected neighbour cells since the HIV-1 receptors are downregulated; 2) harboring cells with reactivated HIV-1 can be recognized by specific CTLs (cytotoxic CD8+ T cells), by NK (Natural Killer) cells and by specific cytotoxic antibodies; and 3) the reactivated HIV-1 will be targeted and neutralized by anti-retroviral therapy that need to be maintained or intensified during the treatment with the combination therapy comprising bryostatins and HDACs inhibitors.

The dosage amount of bryostatin 1 is preferably in the range from 5 and 50 μg/m² /day, more preferably 10-25 μg/m²/day. Infusion times for bryostatin-1 are generally up to 24 h, more preferably 1-3 hours, with 1 h most preferred. Patients will receive a media of 6 intravenous infusions once weekly. Bryostatain-1 will be administered in PET diluent (10 μg/ml of 60% polyethylene glycol, 30% ethanol, 10% Tween 80) via a portable infusion pump. Valproic acid will be given orally (1500 mg/day) and adjusted to maintain plasma concentrations within a defined range (50-100 mg/L). The dosage amount of phenylbutyrate is preferably in the range from 5 to 20 grams/day, more preferably 7.5 to 15 grams/daily. Either Phenylbutyrate or Valproic acid will be given orally and daily during the time of bryostatin-1 treatment. The combination therapy is not restricted to valproic acid and phenylbutyrate and other HDACs inhibitors such as hydroxamic acids (SHA, LAQ-824) and Benzamides (MS-275, CI-994) can be included in the combination therapy.

As noted above, the present invention should be combined with one or more agents useful in the treatment of HIV infection. It will be understood that the scope of combinations of the compounds of this invention with HIV/AIDS antivirals, immunomodulators, anti-infectives or vaccines is not limited to the following list, and includes in principle any combination with any pharmaceutical composition useful for the treatment of AIDS. The HIV/AIDS antivirals and other agents will typically be employed in these combinations in their conventional dosage ranges and regimens as reported in the art.

Suitable antiviral agents include (but not restricted) those listed herein. ANTIVIRALS Manufacturer (Tradename and/or Drug Name Location) Indication (Activity): abacavir Glaxo Welcome HIV infection, AIDS, ARC GW 1592 (ZIAGEN.®.) (nRTI); 1592U89 abacavir+GlaxoSmithKline HIV infection, AIDS, ARC (nnRTI); lamivudine+(TRIZIVIR.®.) zidovudine acemannan Carrington Labs ARC (Irving, Tex.) ACH 126443 Achillion Pharm. HIV infections, AIDS, ARC (nucleoside reverse transcriptase inhibitor); acyclovir Burroughs Wellcome HIV infection, AIDS, ARC, in combination with AZT AD-439 Tanox Biosystems HIV infection, AIDS, ARC AD-519 Tanox Biosystems HIV infection, AIDS, ARC adefovir dipivoxil Gilead HIV infection, AIDS, ARC GS 840 (RTI); AL-721 Ethigen ARC, PGL, HIV positive, (Los Angeles, Calif.) AIDS alpha interferon Glaxo Wellcome Kaposi's sarcoma, HIV, in combination w/Retrovir AMD3100 AnorMed HIV infection, AIDS, ARC (CXCR4 antagonist); amprenavir Glaxo Wellcome HIV infection, AIDS, 141 W94 (AGENERASE.®.) ARC (PI); GW 141 VX478 (Vertex) ansamycin Adria Laboratories ARC LM 427 (Dublin, Ohio) Erbamont (Stamford, Conn.) antibody which neutralizes; Advanced Biotherapy AIDS, ARC pH labile alpha aberrant Concepts (Rockville, Interferon Md.) AR177 Aronex Pharm HIV infection, AIDS, ARC atazanavir (BMS 232632) Bristol-Myers-Squibb HIV infection, AIDS, ARC (ZRIVADA.®.) (PI); beta-fluoro-ddA Nat'l Cancer Institute AIDS-associated diseases BMS-232623 Bristol-Myers Squibb/HIV infection, AIDS, (CGP-73547) Novartis ARC (PI); BMS-234475 Bristol-Myers Squibb/HIV infection, AIDS, (CGP-61755) Novartis ARC (PI); capravirine Pfizer HIV infection, AIDS, (AG-1549, S-1153) ARC (nnRTI); CI-1012 Warner-Lambert HIV-1 infection cidofovir Gilead Science CMV retinitis, herpes, papillomavirus curdlan sulfate AJI Pharma USA HIV infection cytomegalovirus immune MedImmune CMV retinitis globin cytovene Syntex sight threatening CMV ganciclovir peripheral CMV retinitis delavirdine Pharmacia-Upjohn HIV infection, AIDS, (RESCRIPTOR.™.) ARC (nnRTI); dextran Sulfate Ueno Fine Chem. Ind. AIDS, ARC, HIV Ltd. (Osaka, Japan) positive asymptomatic ddC Hoffman-La Roche HIV infection, AIDS, ARC (zalcitabine, (HIVID.®.) (nRTI); dideoxycytidine ddl Bristol-Myers Squibb HIV infection, AIDS, ARC; Dideoxyinosine (VIDEX.®.) combination with AZT/d4T (nRTI) DPC 681 & DPC 684 DuPont HIV infection, AIDS, ARC (PI) DPC 961 & DPC 083 DuPont HIV infection AIDS, ARC (nnRTRI); emvirine Triangle Pharmaceuticals HIV infection, AIDS, ARC (COACTINON.®.) (non-nucleoside reverse transcriptase inhibitor); EL10 Elan Corp, PLC HIV infection (Gainesville, Ga.) efavirenz DuPont HIV infection, AIDS, (DMP 266) (SUSTIVA.®.) ARC (nnRTI); Merck (STOCRIN.®.) famciclovir Smith Kline herpes zoster, herpes simplex emtricitabine Triangle Pharmaceuticals HIV infection, AIDS, ARC FTC (COVIRACIL.®.) (nRTI); Emory University emvirine Triangle Pharmaceuticals HIV infection, AIDS, ARC (COACTINON.®.) (non-nucleoside reverse transcriptase inhibitor); HBY097 Hoechst Marion Roussel HIV infection, AIDS, ARC (nnRTI); hypericin VIMRx Pharm. HIV infection, AIDS, ARC recombinant human; Triton Biosciences AIDS, Kaposi's sarcoma, interferon beta (Almeda, Calif.); ARC interferon alfa-n3 Interferon Sciences ARC, AIDS indinavir; Merck (CRIXIVAN.®.) HIV infection, AIDS, ARC, asymptomatic HIV positive, also in combination with AZT/ddI/ddC (PI); ISIS 2922 ISIS Pharmaceuticals CMV retinitis JE2147/AG1776; Agouron HIV infection, AIDS, ARC (PI); KNI-272 Nat'l Cancer Institute HIV-assoc. diseases lamivudine; 3TC Glaxo Wellcome HIV infection, AIDS, (EPIVIR.®.) ARC; also with AZT (nRTI); lobucavir Bristol-Myers Squibb CMV infection; lopinavir (ABT-378) Abbott HIV infection, AIDS, ARC (PI); lopinavir+ritonavir Abbott (KALETRA.®.) HIV infection, AIDS, ARC (ABT-378/r) (PI); mozenavir AVID (Camden, N.J.) HIV infection, AIDS, ARC (DMP-450) (PI); nelfinavir Agouron HIV infection, AIDS, (VIRACEPT.®.) ARC (PI); nevirapine Boeheringer HIV infection, AIDS, Ingleheim ARC (nnRTI); (VIRAMUNE.®.) novapren Novaferon Labs, Inc. HIV inhibitor (Akron, Ohio); pentafusaide Trimeris HIV infection, AIDS, ARC T-20 (fusion inhibitor); peptide T Peninsula Labs AIDS octapeptide (Belmont, Calif.) sequence PRO 542 Progenics HIV infection, AIDS, ARC (attachment inhibitor); PRO 140 Progenics HIV infection, AIDS, ARC (CCR5 co-receptor inhibitor); trisodium Astra Pharm. Products, CMV retinitis, HIV infection, phosphonoformate Inc other CMV infections; PNU-140690 Pharmacia Upjohn HIV infection, AIDS, ARC (PI); probucol Vyrex HIV infection, AIDS; RBC-CD4Sheffield Med. Tech HIV infection, AIDS, (Houston Tex.) ARC; ritonavir Abbott HIV infection, AIDS, (ABT-538) (RITONAVIR.®.) ARC (PI); saquinavir Hoffmann-LaRoche HIV infection, AIDS, (FORTOVASE.®.) ARC (PI); stavudine d4T Bristol-Myers Squibb HIV infection, AIDS, ARC didehydrodeoxy-(ZERIT.®.) (nRTI); thymidine T-1249 Trimeris HIV infection, AIDS, ARC (fusion inhibitor); TAK-779 Takeda HIV infection, AIDS, ARC (injectable CCR5 receptor antagonist); tenofovir Gilead (VIREAD.®.) HIV infection, AIDS, ARC (nRTI); tipranavir (PNU-140690) Boehringer Ingelheim HIV infection, AIDS, ARC (PI); TMC-120 & TMC-125 Tibotec HIV infections, AIDS, ARC (nnRTI); TMC-126 Tibotec HIV infection, AIDS, ARC (PI); valaciclovir Glaxo Wellcome genital HSV & CMV infections virazole Viratek/ICN (Costa asymptomatic HIV positive, ribavirin Mesa, Calif.) LAS, ARC; zidovudine; AZT Glaxo Wellcome HIV infection, AIDS, ARC, (RETROVIR.®.) Kaposi's sarcoma in combination with other therapies (nRTI); [PI=protease inhibitor nnRTI=non-nucleoside reverse transcriptase inhibitor NRTI=nucleoside reverse transcriptase inhibitor]

### EXAMPLES

The following examples are provided by way of illustration only and not by way of limitation. Those of skill in the art will readily recognize a variety of noncritical parameters that could be changed or modified to yield essentially similar results.

### Example 1

### HPLC Characterization of Bryostatin-1

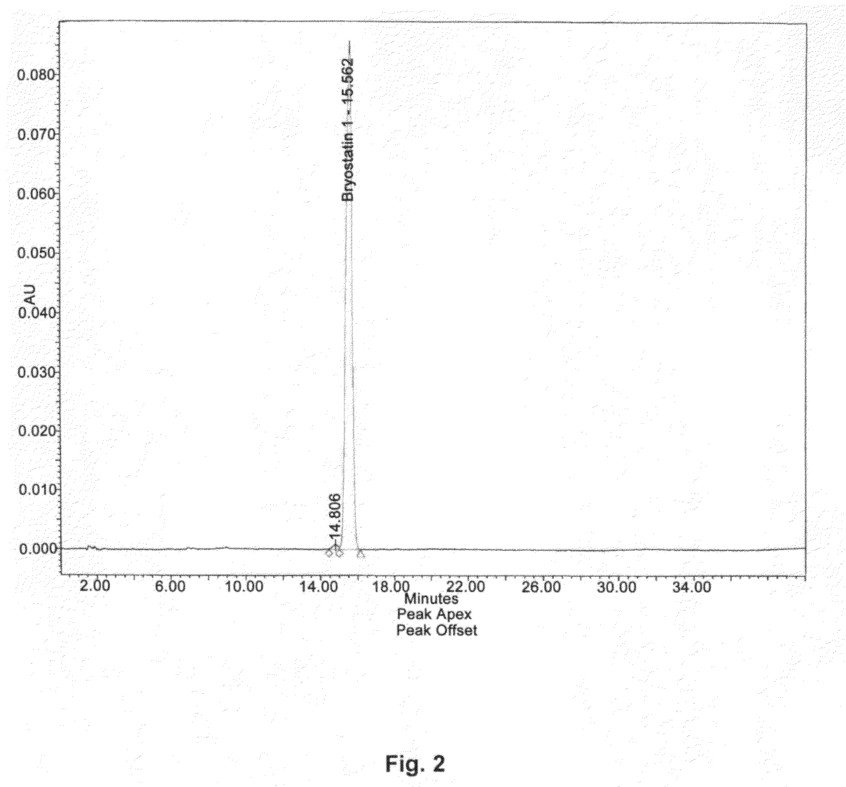

Bryostatin 1 was extracted and purified from Bugula neritina utilizing a supercritical fluid with a polar co-solvent (SuperFluids™) [U.S. Pat. No. 5,750,709, May 12, 1998] followed by downstream chromatographic purification and crystallization. An HPLC chromatogram of the isolated bryostatin 1 is shown in FIG. 2.

### Example 2

### HPLC Characterization of Bryostatin-2

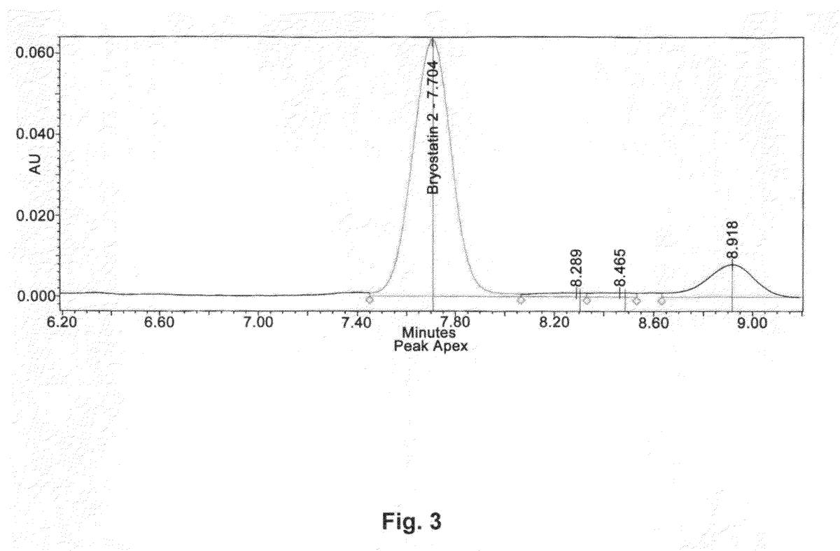

Bryostatin-2 was extracted and purified from Bugula neritina lutilizing a supercritical fluid with a polar co-olvent (SuperFluids™) [U.S. Pat. No. 5,750,709, May 12, 1998] followed by downstream chromatographic purification and crystallization. An HPLC chromatogram of the isolated bryostatin 2 is shown in FIG. 3.

### Example 3

### HPLC Characterization of Bryostatin-3

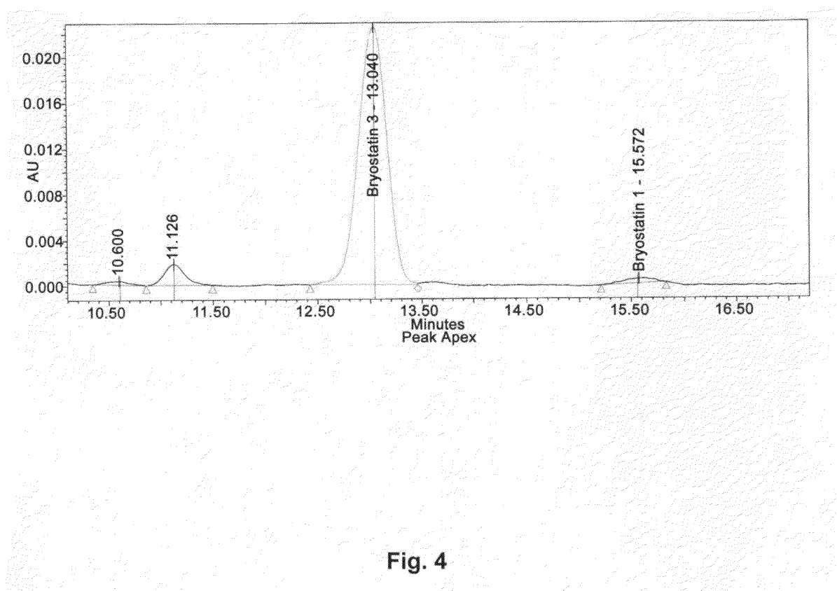

Bryostatin-3 was extracted and purified from Bugula neritina utilizing a supercritical fluid with a polar co-olvent (SuperFluids™) [U.S. Pat. No. 5,750,709, May 12, 1998] followed by downstream chromatographic purification and crystallization. An HPLC chromatogram of the isolated bryostatin 3 is shown in FIG. 4.

### Example 4

### Bryostatin-1 Reactivates HIV-1 Latency in Jurkat-LAT-GFP Cells Generation of Jurkat-LAT-GFP Cells

For the production of viral particles containing an HIV-derived vector, 5×10⁶ 293T cells were transfected with plasmids pEV731 (10 μg), pCMV-R8.91 (6.5 μg) and pcDNA₃-VSV (3.5 μg) in 10 cm dishes. After 16 h, medium was replaced, supernatants containing viral particles were harvested 24 h later and viral particles containing 150 ng of p24 were used to infect 10⁶ Jurkat cells. After 96 h, the efficiency of the infection process was monitored by FACS analysis and the negative population was sorted (FACSCvantage SE, BD Bioscience) and cultured again in completed medium. Then the sorted cells were stimulated with TNFA for 24 h and then the GFP⁺ population was analysed (Cell Quest-Pro software), sorted and cloned by limit dilution in 96 well plates. After three weeks the clones were stimulated with PMA (50 ng/ml) to induce the expression of the integrated LTR-GFP vector for 24 h and 4 out of 72 clones were selected for characterization. The percentage of GFP⁺ cells was analysed by flow cytometry in an EPIC XL flow cytometer (Beckman-Coulter Inc. CA, USA). Ten thousand gated events were collected per sample. Finally, clone 8 was selected for further experiments and renamed Jurkat-LAT-GFP cells.

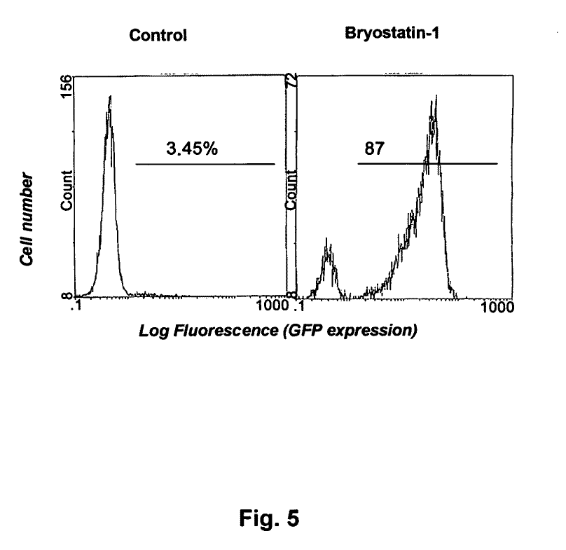

Using the HIV-1 latent cell line Jurkat-LAT-GFP where GFP expression is a subrogate marker of HIV-1 reactivation we found that bryostatin-1 (100 nM) induces HIV-1 reactivation (87% of GFP⁺ cells) (FIG. 5).

### Example 5

### Bryostatins Antagonise HIV-1 Latency in a Concentration Dependent Manner

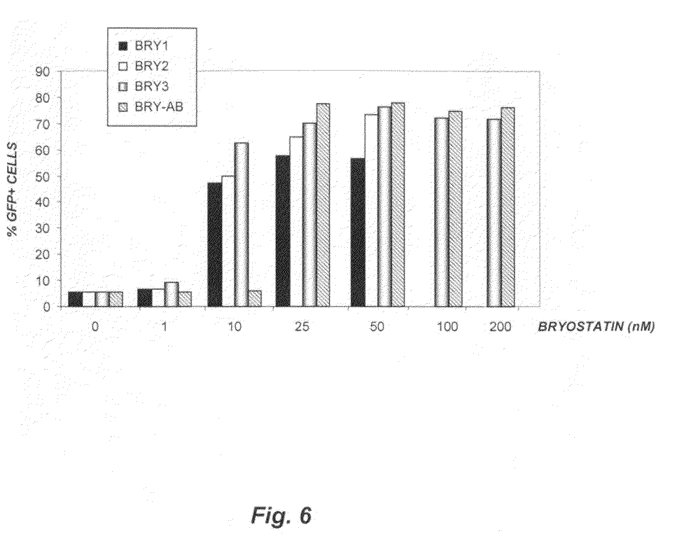

To study the effect of isolated bryostatins Jurkat-LAT-GFP cells were stimulated with increasing concentrations of the compounds for 6 h (FIG. 6). The percentage of GFP⁺ cells was analysed by flow cytometry in an EPIC XL flow cytometer (Beckman-Coulter Inc. CA, USA). Ten thousand gated events were collected per sample.

### Example 6

### Bryostatin-1 is 100 Fold More Potent that Prostratin to Antagonize HIV-1 Latency

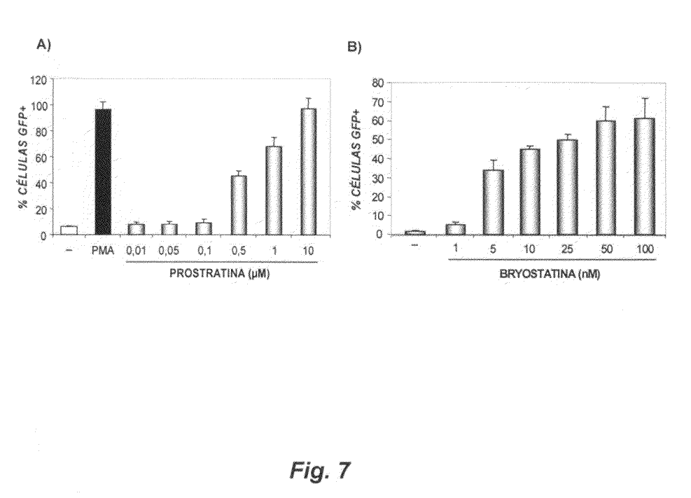

Jurkat-LAT-GFP cells were stimulated with increasing concentrations of the compounds for 6 h (FIG. 7). The percentage of GFP⁺ cells was analysed by flow cytometry in an EPIC XL flow cytometer (Beckman-Coulter Inc. CA, USA). Ten thousand gated events were collected per sample.

### Example 7

### Bryostatin-1 and Prostratin Activates the NF-kB and the MAPKs Pathways with Different Potency

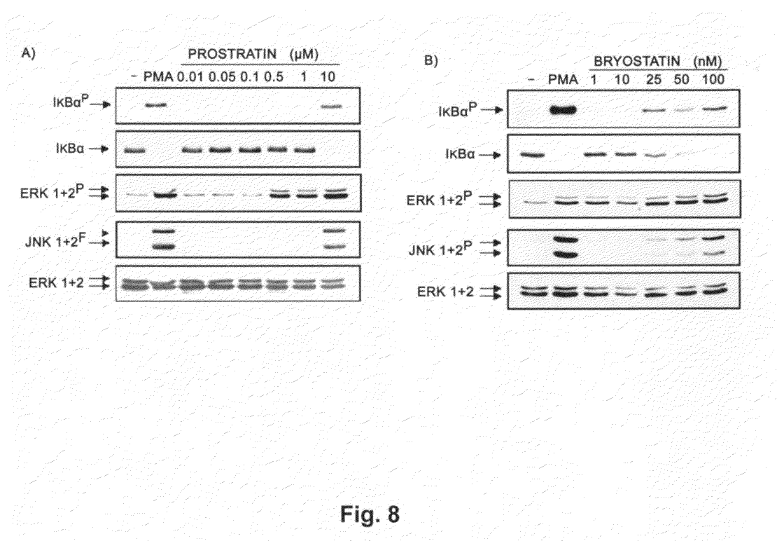

Jurkat LAT-GFP cells were incubated with either bryostatin-1 (1, 10, 25, 50 and 100 nM) or with prostratin (0.01, 0.05, 0.1, 0.5, 1 and 10 μM) for 10 min. IκBα phosphorylation and degradation, the phosphorylation status of MAPKs ERK 1+2 and JNK 1+2, and the steady state levels of total ERK 1+2 were analyzed using specific antibodies by western blots. Control and treated cells were washed with PBS and proteins extracted in 50 μl of lysis buffer (20 mM Hepes pH 8.0, 10 mM KCl, 0.15 mM EGTA, 0.15 mM EDTA, 0.5 mM Na₃VO₄, 5 mM NaFl, 1 mM DTT, leupeptin 1 μg/ml, pepstatin 0.5 μg/ml, aprotinin 0.5 μg/ml, and 1 mM PMSF) containing 0.5% NP-40. Protein concentration was determined by the Bradford assay (Bio-Rad, Richmond, Calif., USA) and thirty μg of proteins were boiled in Laemmli buffer and electrophoresed in 10% SDS/polyacrylamide gels. Separated proteins were transferred to nitrocellulose membranes (0.5 A at 100 V; 4° C.) for 1 h. Blots were blocked in TBS solution containing 0.1% Tween 20 and 5% non-fat dry milk overnight at 4° C., and immunodetection of specific proteins was carried out with primary antibodies using an ECL system (GE Healthcare). The gels are shown in FIG. 8.

### Example 8

### Bryostatin-1 Antagonizes HIV-1 Latency through Classical PKCs- and ERK-Dependent Pathways

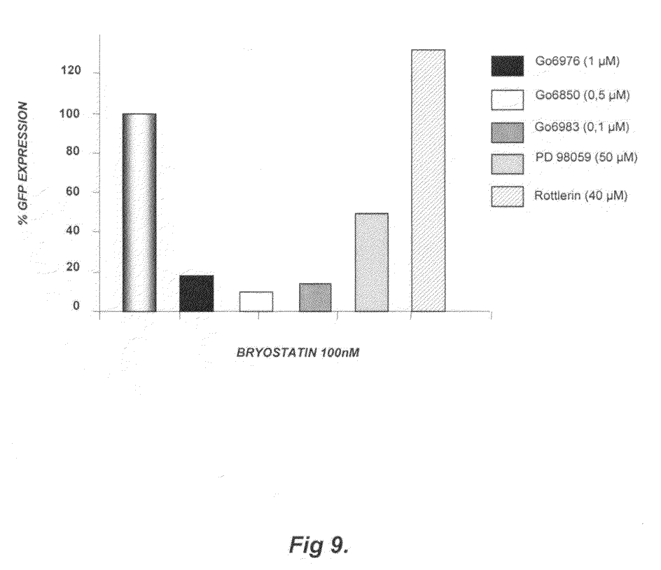

Jurkat LAT-GFP cells were pretreated with the indicated inhibitors for 30 min at the indicated dose, and then stimulated with bryostatin-1 (10 nM) for 6 h. The percentage of GFP+ cells was measured by flow cytometry. Results, shown in FIG. 9, are represented as percentage of activation compared to cells treated with agonists in the absence of the chemical inhibitors (100% activation). The chemical inhibitors Gö6976 (classical PKCs inhibitor), Gö6850 (classical and novel PKCs inhibitor), Gö6983 (pan-PKC inhibitor), rottlerin (PKCδ inhibitor) and PD98059 (MEK inhibitor) were used at the indicated concentrations.

### Example 9

### Synergistic Effects of Suboptimal Concentrations of Bryostatin-1 (1 nM) and HDACs Inhibitors (VPA; 5 mM; TSA; 200 nM) on HIV-1 Reactivation

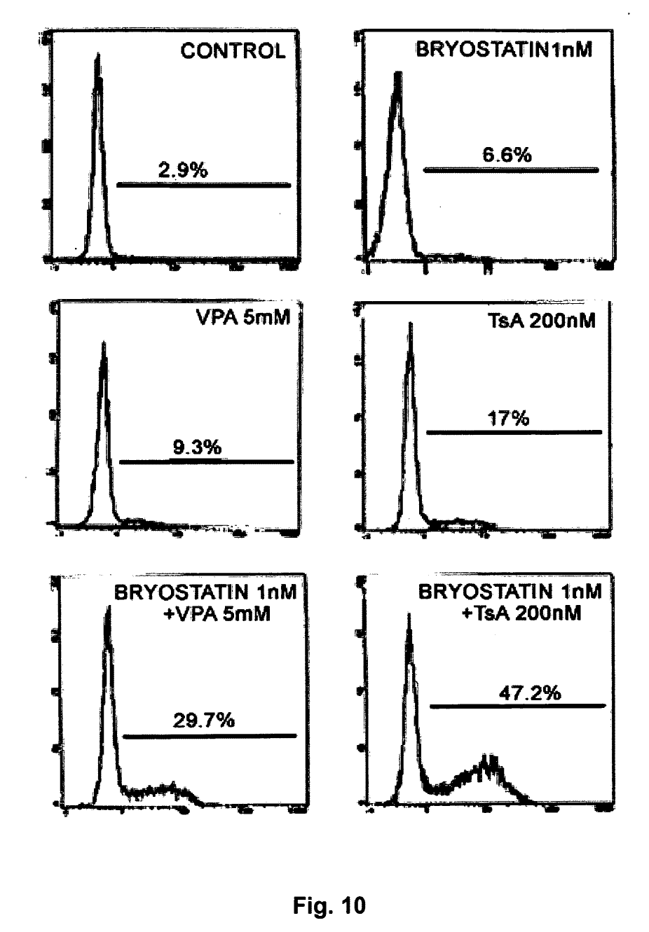

Jurkat-LAT-GFP cells were treated as indicated for 6 h and the percentage of GFP+ cells was measured by flow cytometry (FIG. 10).

### Example 10

### Synergistic Effects of Suboptimal and Optimal Concentrations (1 nM and 10 nM) of Bryostatin-1 and -2, and HDACs Inhibitors (VPA at 1 and 5 mM; TSA at 100 and 200 nM) on HIV-1 Reactivation

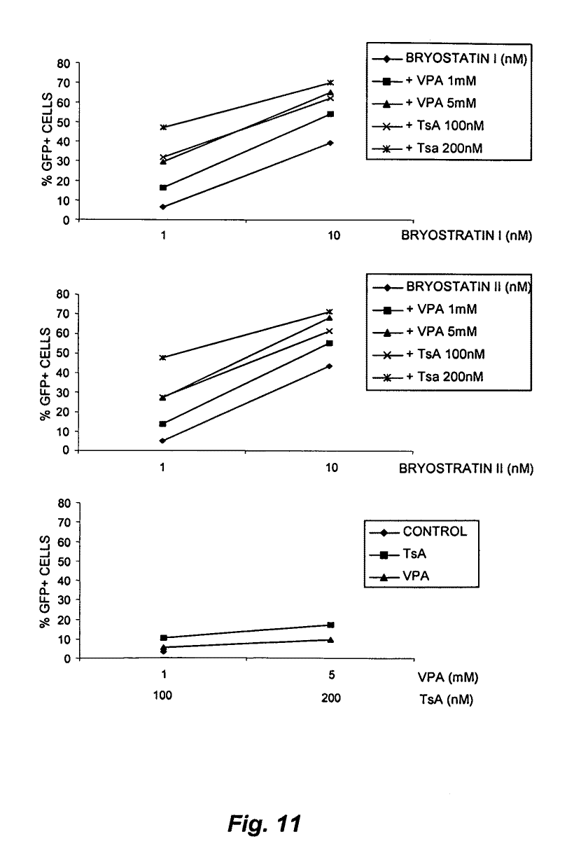

Jurkat-LAT-GFP cells were treated as indicated for 6 h and the percentage of GFP+ cells was measured by flow cytometry. The results are shown in FIG. 11.

### Example 11

### Bryostatin-1 Downregulates the Expression of the HIV-1 Receptors CD4and CXCR4 on the Cell Surface of MT-2 Cells

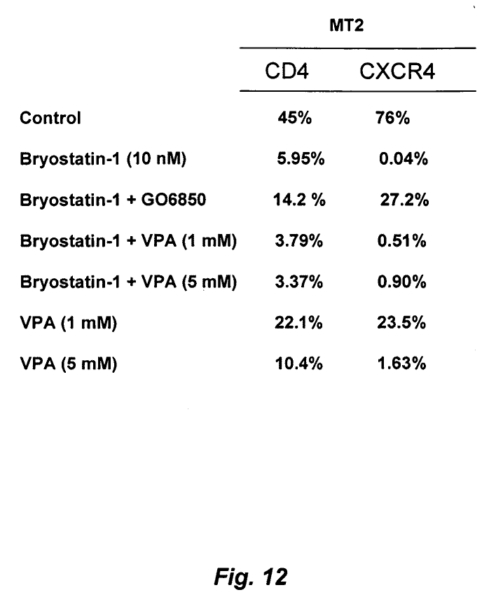

This effect, shown in FIG. 12, is mediated though a PKC-dependent pathway and is not affected by the presence of VPA. MT-2 cells were treated with 10 nM of bryostatin in the presence or absence of either the PKC inhibitor Gö6850 or the HDAC inhibitor VPA for 24 h and the expression of CD4 and CXCR4 analysed. Cell surface expression of CD4 and CXCR4 antigens were measured by direct fluorescence using specific mAbs and analyzed by flow cytometry in an EPIC XL flow cytometer (Beckman-Coulter Inc. CA, USA). The anti-CXCR4 (clone 12G5, PE-labeled) was from BD Biosciences Pharmigen (San Diego, Calif., USA). The mAb anti-CD4 (clone 6D10, FITC-labelled) was from ImmunoTools (Friesoythe, Del.). Dual-Color Reagent Mouse IgG1/FITC+ Mouse IgG1/PE from DAKO (clone DAK-GO1 directed towards Aspergillus niger glucose oxidase) was used as negative control.

### Example 12

### Cytoprotective Effect of Bryostatins on HIV-1-Induced Cell Death in CEM-SS Cells

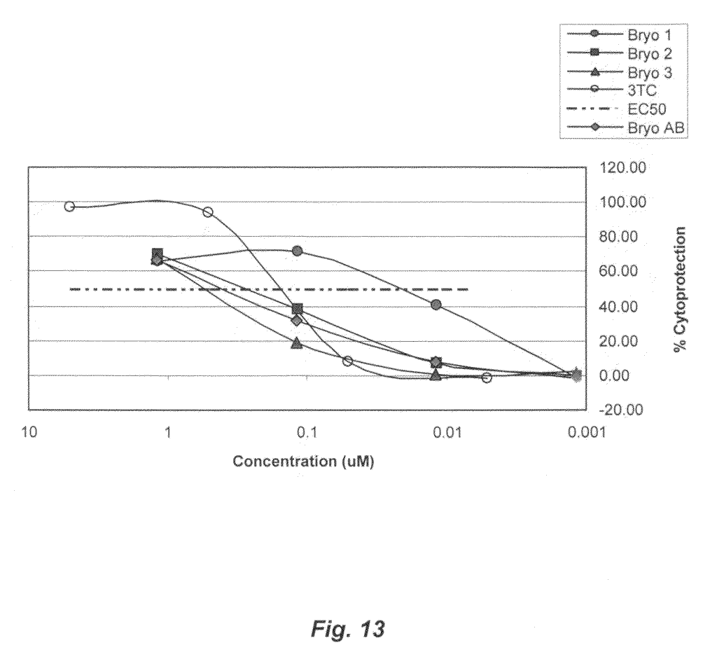

The human T-lymphoblastic cell line CEM-SS was used as the target cell line and virus infections were performed using the HIV_{IIIB} variant of HIV-1 (FIG. 13). Briefly, increasing concentrations of bryostatins (1, 2, 3 and AB) or 3TC (a known inhibitor of HIV RT) were incubated with 5,000 CEM-SS cells and HIV_{IIIB} in a final volume of 200 μl/well at 37° C. for 6 days. After 6 days, 50 μl/well of XTT dye was added and the plate incubated for 4 hours at 37° C. The plate was then read at 450 nm with a reference at 630 nm and the percent CPE, percent inhibition, percent toxicity, effective concentration 50 (EC₅₀), cytotoxic concentration (CC₅₀) and the selective index (CC₅₀/EC₅₀) were calculated. Plates contained the following controls: media, cellular and viral.

What is claimed is the following:

1. A drug for the treatment of HIV/AIDS latency consisting of protein kinase C modulators

2. In the method of claim 1, the protein kinase C modulator is a cyclic macrolide such as bryostatin 1, bryostatin 2, bryostatin 3 and other bryostatins

3. In the method of claim 2, bryostatins can be administered at low concentrations that minimize toxic side-effects

4. In the method of claim 3, bryostatins can be administered at concentrations that would produce a level of around 10 nanomolar in the blood stream.

5. A drug for treating HIV/AIDS latency consisting of a combination of: (i) protein kinase C modulators; and (ii) histone deacetylase inhibitors

6. In the method of claim 5 where the combination of histone deacetylase inhibitors is synergistic with protein kinase C modulators, reducing the required blood concentration of protein kinase C modulator by an order of magnitude to 1 nanomolar

7. In the method of claim 6, histone deacetylase inhibitors consist of compounds such as valproic acid, phenylbutyrate, hydroxamic acids and benzamides

8. In the method of claim 7 where the preferred histone deacetylase is valporic acid and TSA

9. A drug for treating HIV/AIDS latency consisting of a combination of: (i) protein kinase C modulators; (ii) histone deacetylase inhibitors; and (iii) antivirals.

10. In the method of claim 9, the antivirals are for the treatment of HIV/AIDS.

11. In the method of claim 10, the antivirals consist of one or more of the following: (i) protease inhibitors; (ii) nucleoside reverse transcriptase inhibitors; (iii) fusion inhibitors; (iv) integration inhibitors; (v) CCR5 co-receptor inhibitors and/or (vi) maturation inhibitors.

12. A drug for treating HIV/AIDS latency consisting of a combination of: (i) protein kinase C modulators; (ii) histone deacetylase inhibitors; (iii) antivirals; and (iv) pharmaceutical acceptable carriers for improving drug stability and delivery.

13. In the method of claim 12, pharmaceutical acceptable carriers include: (i) nanoparticles of the drugs; (ii) encapsulation of the drugs in phospholipids nanosomes; and/or (iii) biodegradable polymer nanospheres.
